(19) United States
(12) Patent Application Publication
(10) Pub. No.: US 2021/0298387 A1
(43) Pub. Date: Sep. 30, 2021

(54) PROTECTIVE MASK ACCESSORIES

(71) Applicant: Mario M. De La Guardia, Fernandino Beach, FL (US)

(72) Inventor: Mario M. De La Guardia, Fernandino Beach, FL (US)

(21) Appl. No.: 17/135,359

(22) Filed: Dec. 28, 2020

Related U.S. Application Data
(60) Provisional application No. 63/013,306, filed on Apr. 21, 2020.
(60) Provisional application No. 63/003,010, filed on Mar. 31, 2020.

(51) Int. Cl. A41D 13/11; A62B 23/02

(52) U.S. Cl. CPC A41D 13/1184; A62B 23/025; A41D 13/1161

(57) ABSTRACT

Face-worn accessories provide protection against air-borne particulates. A curved rigid or semi-rigid plastic frame with inner and outer surfaces surrounds an open window covered by a curved, transparent plastic shield. The upper and side portions of the frame include a compressible seal. The frame is worn over the mouth and nostrils of a wearer, with opposing side elastic members extending around the ears of a wearer, such that the compressible seal is conformal to a wearer's face. A gap at the lower portion of the frame is covered with filtration material, which may be provided by an existing removable, replaceable facial mask with the around-the-ear flexible bands of the existing mask being used to hold the entire accessory in position. The invention affords, greater visualization, improved protection with enhanced breathing, while allowing the user to chose their own mask or material for filtration purposes.

### REFERENCE TO RELATED APPLICATIONS

This application claims priority to, and the benefit of, U.S. Provisional patent application Ser. No. 63/003,030, filed Mar. 31, 2020, and U.S. Provisional patent application Ser. No. 63/013,306, filed Apr. 21, 2020, the entire content of all related applications being incorporated herein by reference.

### FIELD OF THE INVENTION

This invention relates generally to face shields, respirators, and the like, and, in particular, to accessories for protective masks providing enhanced effectiveness and visualization.

### BACKGROUND OF THE INVENTION

Various protective masks or “respiratory filters” are commercially available. Such products come in a range of ratings that relate to how the respirator performs (i.e., how much it filters) and in what environment it is designed to be used. By convention, masks designations use a letter followed by a number. The Respirator Rating Letter Classes are N, for Not Oil Resistant; R for Resistant to Oil; and P for Oil Proof.

The Respirator Rating Number Class designates the percentage of particles at least 0.3 microns in diameter that the filter material removes. Thus 95 means the mask removes 95% of all particles that are at least 0.3 microns in diameter. A rating of 100 is reserved for HE or HEPA quality filters which removes 99.97% of all particles that are 0.3 microns in diameter or larger.

As number rating increases, filtration is more effective, but breathing fatigue will increase as well. While it can be critical to avoid choosing a rating that is too low, a 100 filter rating is not necessary for every application. Typically, a wearer will find an 95 rated respirator cooler, and easier to breath through as compared to a 99 rated mask, for example.

The type of mask most commonly used for flu or Influenza prevention is the N95 mask. According the Centers for Disease Control (CDC), these masks are effective in helping to prevent the spread of flu by eliminating transmission from person to person.

N95 masks are designed to fit over the nose and mouth of the wearer, and properly fitted can provide adequate protection. However, existing masks are opaque, such that others cannot see facial features, which may be important for communication, especially in emergency situations. Proper fitting is also difficult to achieve. Since existing masks are typically made of filter material with small bendable tabs, gaps often remain around the face. Further, most existing masks typically leave the eyes exposed.

### SUMMARY OF THE INVENTION

Face-worn accessories provide protection against air-borne particulates, including viruses, bacteria and other pathogens, and dust, smoke, and other contaminants. The accessory includes a curved rigid or semi-rigid plastic frame with inner and outer surfaces defined by an upper portion, a lower portion and two side portions surrounding an open window which is covered by a curved, transparent plastic shield. A compressible sealing member is supported on the inner surfaces of the upper and side portions of the frame. The frame is worn over the mouth and nostrils of a wearer, with opposing side elastic members extending around the ears of a wearer, such that the compressible seal is conformal to a wearer's face. The structure when worn leaves an open gap between the lower portion of the frame around a wearer's chin area is covered by a layer of filtration material.

The layer of filtration material may be provided by an existing facial mask having around-the-ear flexible bands, and the flexible bands may be used to the hold the frame and shield in position. The side portions of the frame may include band-retaining structures, which may be used to retain the bands of the existing facial mask which are looped around the ears of a wearer.

In preferred embodiments, the transparent shield is a removable and replaceable this sheet of flexible plastic that snaps onto the frame. For this purpose, the side portions of the frame may include shield-retaining clips or other structures, enabling the shield to removably fasten onto the frame. The shield has a height greater than the upper portion of the frame, such that the shield overlaps the eyes of a wearer. Alternatively the shield may be more coextensive with the periphery of the frame. Preferred embodiments further include a chin rest extending from the inner surface of the lower portion of the frame to keep the shield spaced-apart from a user's face.

The compressible seal around the inner portion of the top and sides of the frame may comprise an open- or closed-cell foam. The compressible seal may also include air-filled pockets.

### BRIEF DESCRIPTION OF THE DRAWINGS

FIG. 1 is a side perspective view of a preferred embodiment of the invention without a filter coupled thereto;

FIG. 2 is a view seen from the inside of the accessory, without a filtration mask attached, but showing sealing material around the top and sides of the article.

FIG. 3 is a side view showing the way in which the accessory is worn by a user with an existing mask for air filtration;

FIG. 4 is a front view showing an alternative face shield;

FIG. 5 is a front view showing a different alternative face shield; and

FIG. 6 is a front view showing yet a further alternative face shield.

### DETAILED DESCRIPTION OF THE PREFERRED EMBODIMENTS

This invention improves upon the art of filtration masks and face shields by providing accessories that do not obscure a user's face, and enable a user to wear a mask of their choosing for filtration purposes. The accessory, which may be reused, offers numerous advantages, including easier breathing and improved communication in conjunction with a user's choice of suitable filter material(s), while allowing masks incorporating HEPA and other materials to be used for protection against air-borne pathogens, viral and bacterial infection, dust and other contaminants.

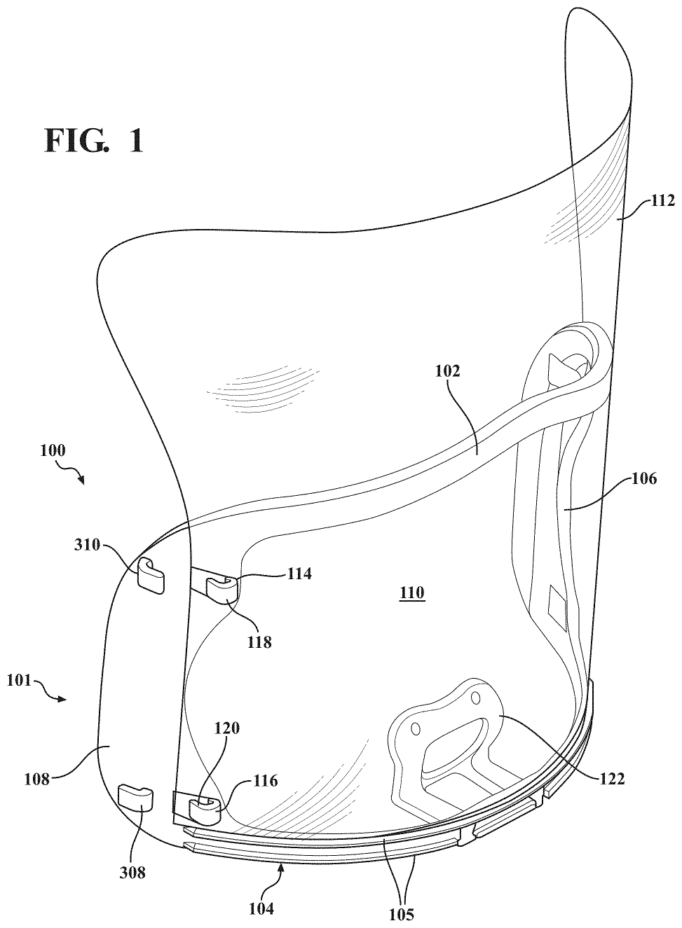

Now making reference to the accompanying drawings, FIG. 1 is an oblique, perspective view of a preferred embodiment of the invention depicted generally at 100. The article broadly includes a rigid or semi-rigid curved plastic frame 101 configured to cover at least the lower nose portion and mouth of a wearer. The frame 101 comprises upper, lower, and side members 102, 104, 106, 108 forming an open window 110 configured for general disposition over the mouth of a wearer. The lower member 104 may have outwardly extending ridges 105 to better grip an existing mask, as discussed below.

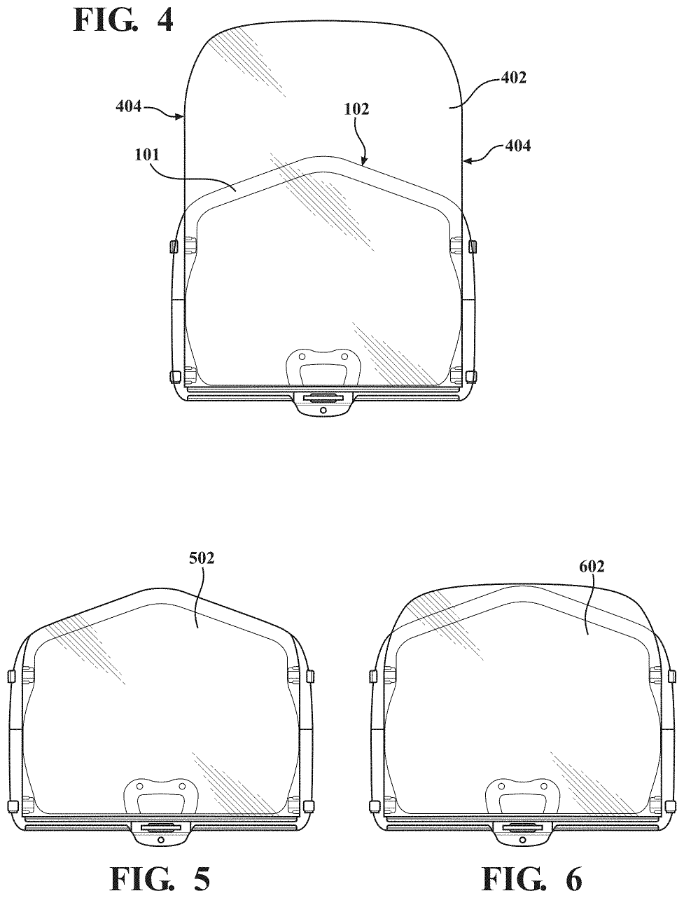

The open window 110 of frame 101 is covered by a curved, snap-on transparent plastic shield 112. FIGS. 1-3 show a tall shield embodiment of the invention, which shields the eyes of the wearer. As shown in FIGS. 4-6, however, the shield may be shorter without covering the eyes. The different interchangeable shields have side apertures (i.e., 114, 116) which are received by clips (i.e., 118, 120) on the frame 101, maintaining the shield in position. While this clip/aperture system is preferred, any suitable shield mounting arrangement may instead be used.

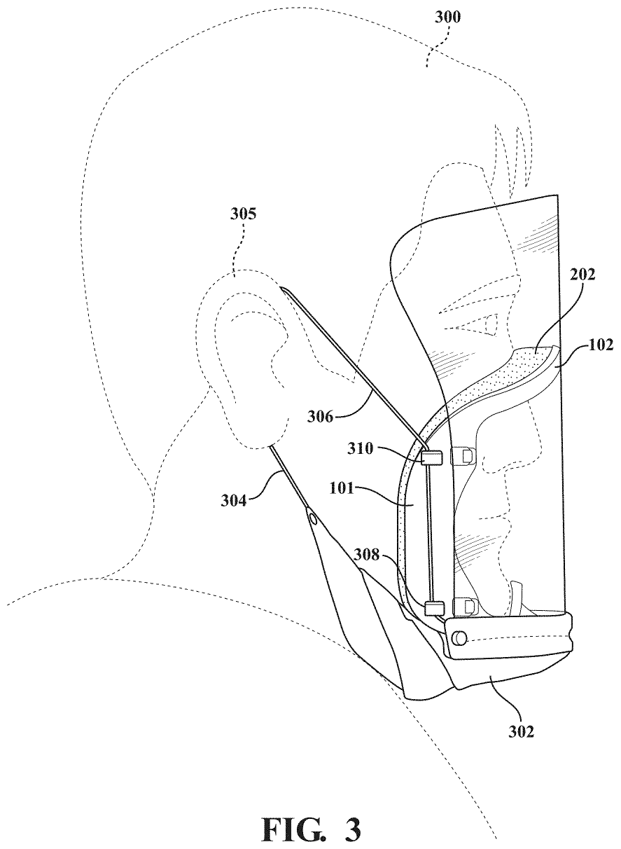

The preferred embodiments further include a snap-on plastic chin stand-off element 122. This component 122 is used to keep the frame and shield away from the face of a wearer when the elastic bands of the filtration mask are dressed over the ears as shown in FIG. 3.

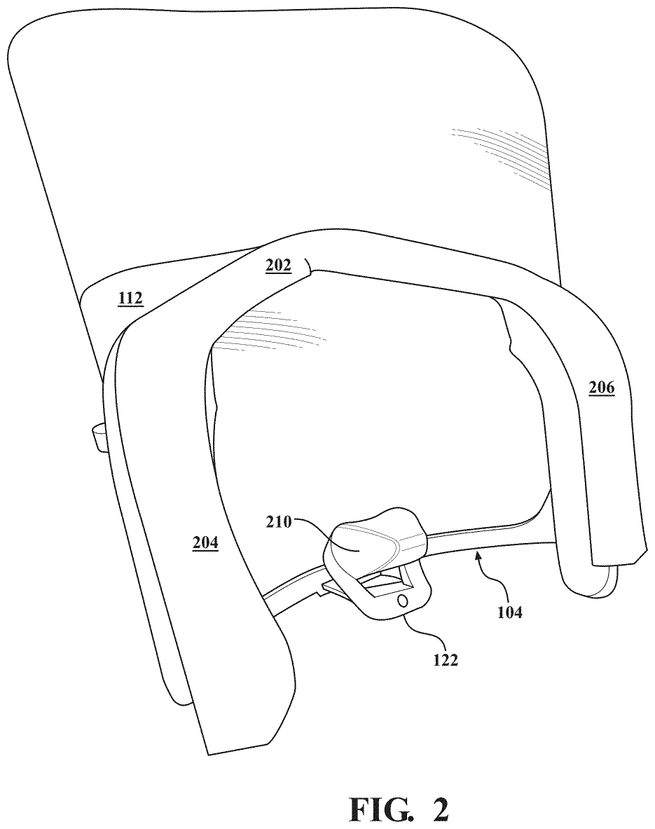

FIG. 2 is a view seen from the inside of the accessory, without a filtration mask attached, but showing sealing material around the top 202 and sides 204, 206 of the article. In the preferred embodiment, the snap-on plastic chin stand-off element 122 is also provided with cushioning material 210. These sealing member(s) may be of any suitable, compressible, face-conformal construction, including closed/open cell foam or any of the chambered members disclosed is the '306 provisional application referenced above and incorporated herein by reference.

FIG. 3 is a side view showing the way in which the accessory is worn by a user with an existing mask for air filtration. In particular, a conventional paper/fabric mask 302 is shown coupled to the frame 101 to seal off the bottom portion thereof. The mask 302 has opposing elastic bands (i.e., 304) that surround the ears (305) of a wearer 300. The right band 304 has an upper portion 306 that is clipped to a lower clip 308 and an upper clip 310 on frame 101. The lower portion of the band 304 is pulled further back under the ear 305, thereby closing off the open bottom of the frame. The upper edge of the existing mask 302 is pulled against the lower member 104 of the frame 101, with ridge(s) 105 enhancing the seal between the mask and the frame. The clips 308, 310, which are on both side of the frame, may simply comprise small plastic curled tabs that hold the string of the existing mask in position as shown.

An advantage of the invention is that any type of filtration material may be used to cover the bottom portion of the frame. This would include, without limitation, existing flexible paper, fiber, and fabric N95 masks with opposing side elastic bands or cords like the one depicted in FIG. 2. However, the invention is not limited in this respect, as other materials with higher/lower filtration values may be used. If the filter material does not have side bands or if they are not long enough, additional elastic band or cords may be used from the frame 101 around a user's ears on both sides. In all embodiments disclosed herein, the actual air filtration portion is preferably disposed on a bottom surface away from the user's features. This not only results in enhanced visualization, but such placement also helps prevent air-borne particulate transfer.

FIGS. 4-6 illustrate alternative snap-on, removable and replaceable face shield shapes. Specifically, the shield 402 of FIG. 4 extends above the upper portion 102 of the frame 101, but has straight sides 404 as opposed to opposing side wings around the eyes of the wearer. In FIG. 5, the periphery of the shield 502 is generally coextensive with the outer edge of the frame, whereas in FIG. 6, the shield 602 extends slightly above the upper portion of the frame.

Any suitable plastic material may be used to construct the frame and shield, including polyethylene, ABS, vinyl, polycarbonate, and so forth. The frame is preferably injection molded, whereas the shield is die cut from sheet material.

1. A face-worn accessory providing protection against air-borne particulates including pathogens and contaminants, comprising:
a curved rigid or semi-rigid plastic frame with inner and outer surfaces defined by an upper portion, a lower portion and two side portions surrounding an open window;
a compressible seal on the inner surfaces of the upper and side portions of the frame;
wherein the frame is adapted to worn over the mouth and nostrils of a wearer, with the compressible seal being conformal to a wearer's face but leaving an open gap between the lower portion of the frame and a wearer's chin area;
a curved, transparent plastic shield configured to cover the open window of the frame; and
wherein the open gap is adapted to be covered by a layer of filtration material.

2. The face-worn accessory of claim 1, wherein the layer of filtration material is provided by an existing paper or fabric facial mask.

3. The face-worn accessory of claim 1, wherein:
the layer of filtration material is provided by an existing paper or fabric facial mask having around-the-ear flexible bands; and
the flexible bands are used to the hold the frame against a wearer's face.

4. The face-worn accessory of claim 1, wherein:
the side portions of the frame includes band-retaining structures;
the layer of filtration material is provided by an existing paper or fabric facial mask having around-the-ear flexible bands; and
the band-retaining structures are used to retain the bands of the existing paper or fabric facial mask and hold the frame against a wearer's face.

5. The face-worn accessory of claim 1, wherein:
the side portions of the frame includes shield-retaining structures; and
the shield-retaining structures enable the shield to removably clip onto the frame.

6. The face-worn accessory of claim 1, wherein the shield has a height greater than the upper portion of the frame such that the shield overlaps the eyes of a wearer.

7. The face-worn accessory of claim 1, further including a chin rest extending from the inner surface of the lower portion of the frame to keep the shield spaced-apart from a user's face.

8. The face-worn accessory of claim 1, wherein the compressible seal is an open- or closed-cell foam.

9. The face-worn accessory of claim 1, wherein the compressible seal includes air-filled pockets.
